(19) United States
(12) Patent Application Publication
(10) Pub. No.: US 2023/0023795 A1
(43) Pub. Date: Jan. 26, 2023

(54) METHOD AND EQUIPMENT FOR MEASURING MOVEMENT OF SUBSTANCES/BOLUS IN A TUBULAR ORGAN

(71) Applicant: ALACER INDUSTRIA ELETRÔNICA LTDA., Sao Paulo (BR)

(72) Inventors: Daniel Mello Moraes Gualberto, Sao Paulo (BR); Fernando F. Carvalho, Sao Paulo (BR)

(21) Appl. No.: 17/812,928

(22) Filed: Jul. 15, 2022

(30) Foreign Application Priority Data
Jul. 15, 2021 (BR) 102021013945-5

(51) Int. Cl. A61B 5/00; A61B 5/0538

(52) U.S. Cl. CPC A61B 5/4233; A61B 5/0538; A61B 5/6852; A61B 5/7257; A61B 5/742; A61B 2562/0209; A61B 2562/043

(57) ABSTRACT

This patent application provides a method and equipment for promoting measurement of movement of substances/bolus in a tubular organ and for use in the impedance phase reading in the esophagus, thus integrating the field of exams performed for medical diagnoses, particularly for carrying out in the esophagus. The method is characterized by taking the difference between tension and current (impedance phase) as a parameter in the Impedance audiometry exam, from an excited element with alternate current; the proposed method uses a probe (5) defined by a flexible insulating catheter (1), including metallic rings (2) that is introduced in the organ (3) to be examined; the metallic rings (2) are connected by wires (4) that run internally through the probe (5) defined by the flexible insulating catheter (1); the probe (5) is connected to an equipment (6) that applies an electric excitation to the metallic rings (2) which act as electrodes.

### CROSS-REFERENCE TO RELATED APPLICATIONS

This application claims priority under 35 U.S.C. § 119 to Brazilian Application No. 102021013945-5 filed Jul. 15, 2021. The contents of the aforementioned application are incorporated herein by reference.

### BACKGROUND INFORMATION

### 1. Field

This patent application provides a method and equipment for promoting, in general, the measurement of movement of substances/bolus in a tubular organ and, more specifically, for use in the impedance phase reading in the esophagus, thus integrating the field of exams performed for medical diagnoses, particularly in the field of exams carried out in the esophagus.

This specification refers to an invention patent application that addresses new methods and equipment especially developed for exam assessment of the movement of substances/bolus inside tubular organs of the human body, the main application of said solution being the use in gastroesophageal exams as an enhancement of the currently used method of Impedance audiometry.

The method and equipment proposed herein are intended for enabling the electric impedance phase reading inside tubular organs of a patient's body (especially, but not exclusively, the esophagus), thus allowing the use of the phase parameter as subsidiary information of other related exams and belonging to the state of the art.

### 2. Background

Impendance audiometry is a diagnostic technique developed in the Helmholtz Institute in Aachen—Germany, in the early 1990s. It should be noted that Impedance audiometry is nowadays one of the most important diagnostic methods.

The first work that featured this technique is the Silny J. Intraluminal multiple electric impedance procedure for measurement of gastrointestinal motility. J Gastrointest Motil 1991; 3:151-62.

This technique/device is also featured in a patient (US) 5,109,870, called “Apparatus for and Method of Motility and Peristalsis Monitoring”.

As featured in said US Patent, “The main function of many organs includes transport of its content through itself. The analysis of the characteristics of this transport, in other words, motility and peristalsis, has important diagnostic value”.

Currently, the most used methods for measuring the motility and peristalsis are Impedance audiometry, Manometry and image diagnosis measurement (namely Fluoroscopy).

In this peristalsis efficiency measurement role, Impedance audiometry is one of the main diagnostic methods, alongside Manometry. There is also another important application for Impedance audiometry, which is the extended monitoring (normally for a 24-hour period) of the esophagus. In this model, not only peristalsis may be monitored, but also reverse transportation (gastroesophageal reflux).

The Gastroesophageal Reflux Disease (GERD) affects 8% to 33% of world population, from all age brackets and both genders, “El-Serag H B, Sweet S, Winchester C C, et al. Update on the epidemiology of gastroesophageal reflux disease: a systematic review. (Gut 2014; 63:871-80)”, and causes estimated expenses higher than 9 to 10 billion dollars/year only in the USA, mainly associated with proton pump inhibitor treatment (stomach acidity reducers) and medical diagnoses “(Shaheen N J, Hansen R A, Morgan D R, et al. The burden of gastrointestinal and liver diseases, 2006. Am J Gastroenterol 2006; 101:2128-38)”.

In the GERD diagnosis, the most used exam is pH monitoring, where an equipment connected to a thin catheter introduced through the nostril monitors and records esophageal pH for 24 hours.

With the advent of Impedance audiometry, multiple impedance sensors were added to this catheter. In this exam model, impedance is also monitored for 24 hours, in addition to pH. With this, diagnostic accuracy has increased, as it is also possible to monitor, in addition to pH (acidity), gas refluxes, non-acidic or low acidity refluxes, swallowing and electric mucosal permeability, which is correlated to its integrity “(Chenxi Xie, Sifrim D, Yuwen Li, Yinglian Xiao. Esophageal Baseline Impedance Reflects Mucosal Integrity and Predicts Symptomatic Outcome With Proton Pump Inhibitor Treatment, J Neurogastroenterol Motil 2018; Vol. 24 No. 1, January)”.

These exams are carried out in outpatient care, where the patient receives a recorder and is discharged for the daily activities. At the end of the exam, they return to the medical facility for removal of the catheter and the examiner reads and interprets the data collected.

Nowadays, pH and impedance monitoring is considered the golden standard for GERD (Gastroesophageal Reflux Disease) diagnosis.

Impedance audiometry basically consists in measuring the movement of material (bolus) in a tubular organ (normally the esophagus) through measurement of electric conductivity. For this objective, several conductive electrodes are used (normally stainless steel rings) applied over an insulating material (plastic catheter), between which a diminutive alternated electric current is applied. The potential difference between said electrodes is measured to determine the conductivity of the medium in which they are inserted (this bolus in the esophagus).

This technique may be applied in any tubular organ (for example, intestine), but only the esophagus will be referenced hereinafter, which is currently the most common application.

When the electrodes are surrounded by air, the current cannot go through, or will face difficulties to pass through, and an increase in electric impedance will be experienced (resistance to current passage or simply electric resistance). When the electrodes are surrounded by any ion-rich content (saliva, mucus, stomach acid, etc.), the current flows more easily, which will lead to a reduction in observed impedance.

It should also be highlighted that the esophageal wall itself is able to allow passage of electric current through its layers (mucosa, submucosa and muscle tissue), therefore, even in the absence of a bolus, a baseline impedance is experienced.

In the physical assembly of the current technique, such as shown in FIG. 1, a catheter is introduced in the esophagus (normally through a nostril). This catheter is made of insulating material (normally plastic) and has exposed metallic conductive rings (electrodes). These rings are connected to wires, which run internally (therefore insulated from the medium) and are connected to the Impedance audiometry equipment.

The equipment applies an electric stimulus to the electrodes, and an electric current shall pass through the contents of the esophagus. By determining the ratio between voltage amplitudes and stimulus current, the equipment determines the resulting impedance between electrodes (classic formula |Z|=|V|/|I|) and stores and/or sends this information for visualization.

The data are shown to the user in graphic format, normally Cartesian charts, but nowadays Clouse plots are also used. The stimulus is carried out in an alternate current, and when |V| and |I| are referred, the applied and measured wave amplitudes are referenced.

### SUMMARY

An embodiment of the present disclosure provides a method and equipment for promoting, in general, the measurement of movement of substances/bolus in a tubular organ and, more specifically, for use in the impedance phase reading in the esophagus, thus integrating the field of exams performed for medical diagnoses, particularly in the field of exams carried out in the esophagus. The method proposed is characterized by taking the difference between tension and current (impedance phase) as a parameter in the Impedance audiometry exam, from an excited element with alternate current; the proposed method uses a probe (5) defined by a flexible insulating catheter (1), including metallic rings (2) that is introduced in the organ (3) to be examined; the metallic rings (2) are connected by wires (4) that run internally through the probe (5) defined by the flexible insulating catheter (1); the probe (5) is connected to an equipment (6) that applies an electric excitation to the metallic rings (2) which act as electrodes.

The features and functions can be achieved independently in various embodiments of the present disclosure or may be combined in yet other embodiments in which further details can be seen with reference to the following description and drawings.

### BRIEF DESCRIPTION OF THE DRAWINGS

The object addressed in this Invention patent application shall be fully understood in depth through the detailed description which will be carried out based on the figures listed below, in which:

FIG. 1 illustrates the physical assembly of the current technique, in which a catheter is introduced in the esophagus of a patient (normally through the nostril), said catheter being made of insulating material (normally plastic) including exposed metallic conductive rings (electrodes); said FIG. 1 indicates the “Current Lines” through the text reference (T01); the “Stimulus Current” through a text reference (T02); and the representative block of the “viewing and/or storage” condition through text reference (T03);

FIG. 2 schematically shows a typical Impedance audiometry exam, where multiple electrodes are placed along the catheter, which enables determining the direction of the substance/bolus inside the patient's esophagus, and the data acquired by the probe and passed over to the equipment and related to the bolus movement featured in a display (D); said FIG. 2 indicates the “Bolus movement” through text reference (T04); the condition according to which the “Equipment stimulates and reads the amplitude of impedances” through text reference (T05); the condition according to which the “Display (D) shows charts to the examiner” through text reference (T06); as well as zones “Z1” to “Z5” established along the patient's esophagus and corresponding zones “Z1” to “Z5” shown in the display (D);

FIG. 3 illustrates the relationship between Cartesian and polar representations of a complex number (impedance).

FIGS. 4 and 4A sequentially illustrates two charts obtained by swallowing monitoring, where the chart featured in FIG. 4 the impedance amplitude is addressed, while the following chart in FIG. 4A features its phase record (Phase delay);

FIG. 5 illustrates an equipment schematic proposed herein in its multiple channel configuration sharing rings, to which derives from the physical assembly belonging to the state of the art and is shown in FIG. 1;

FIG. 5A illustrates a functional diagram of the equipment proposed herein;

FIG. 5B illustrates a functional diagram of the equipment addressed herein with phase measurement with sinusoidal excitation by current or tension;

FIG. 6 illustrates and displays the difference between current and tension in an element excited with sinusoidal current, and its interaction with the phase; FIG. 6 also addresses the phase determination method through measurement of the “zero crossing”;

FIGS. 7A, 7B and 7C illustrate logical key diagrams which integrate the methodology of the equipment proposed herein; and

FIG. 8 illustrates a chart resulting from the square wave current excitation of capacitive charges within the concept of the object proposed in this invention patent application;

### DETAILED DESCRIPTION

Although Impedance audiometry is acknowledged today as a widely useful exam, it is difficult to interpret and may not yield useful readings in some patients.

When diagnosing GERD, 24-hour exams are used, in which the patient receives a recorder and the attached catheter, and may carry on with their day-to-day activities. These are, therefore, extensive records in which the patient will be moving and performing everyday activities. The probe movement inside the patient is expected to cause random impedance variations.

The examiner is responsible for analyzing the entire record and perform separation between artifacts and typical swallowing patterns and refluxes, a task which requires focused attention and concentration, and may take a few hours in more complex cases.

In addition, there is an interpretation variability between examiners, therefore, the same record analyzed by different people leads to results with key differences. This variability negatively affects the determination of normative values and decreases the diagnostic accuracy of the exam.

There are also patients that have a very low baseline mucosal impedance. In such cases, it is very difficult, if not impossible, to determine impedance drops which would indicate the presence of a bolus, partially invalidating the use of this diagnosis tool.

Nowadays, computerized analysis algorithms are being improved, including with the use of artificial intelligence, in order to circumvent the issue of analysis time, but no satisfactory solutions were reached yet.

The Impedance audiometry technique currently used does not allow and does not provide the reading possibility of the impedance phase.

Considering the particularities of the aforementioned state of the art, one of the objectives of this Invention patent application is to propose a “METHOD AND EQUIPMENT FOR MEASURING MOVEMENT OF SUBSTANCES/BOLUS IN A TUBULAR ORGAN”, which allow measurement and evaluation of substances/bolus inside tubular organs of the human body, the gastroesophageal exams being its main application as an enhancement of the currently used Impedance audiometry technique.

It is also another objective of this invention patent application enhancing Impedance audiometry exams and allowing visualization of phenomena that are not visible through conventional techniques.

Other objectives of this invention patent application are enhancing Impedance audiometry exams, facilitating visualization of phenomena of difficult comprehension by standard techniques, such as Impedance audiometry exams, allowing software to analyze results in a more accurate manner, either through deterministic methods or artificial intelligence, and also decreasing the inter and intra-examiner variability of the Impedance audiometry exam.

Lastly, additional objectives of this Invention patent application are improving diagnostic accuracy of Impedance audiometry exams and also decreasing the costs of these exams by increasing precision and decreasing human time required for analysis.

According to the features illustrated by the aforementioned Figures, this invention patent application which proposes a “METHOD FOR MEASURING MOVEMENT OF SUBSTANCE/BOLUS IN A TUBULAR ORGAN” to promote electric impedance phase reading in the esophagus which are based in the principle according to which the main objective of Impedance audiometry is to measure the movement of the substance/bolus in a tubular organ, considering that nowadays the main application of this technique is diagnosing esophageal dysfunctions.

The current technique uses impedance amplitude as a marker for identification of movement. Through this technique, substance/bolus movements may be observed with electrical conductivity different than the medium (baseline esophageal impedance). In general, fluids (saliva, gastroesophageal refluxes) have lower impedance than the baseline esophageal impedance and gases have a very high impedance (ending up interrupting current flow altogether).

This invention features a new technique for studying transport of substances/bolus inside tubular organs (mainly, but not limited to, the esophagus).

In this invention, the same probe already used in the standard Impedance audiometry examination is used, but connected to an equipment that is able to perform readings/store/show not only the impedance amplitude, but also the phase of said impedance.

A Brief Explanation on Impedance Phases

It is known that, mathematically, a measured impedance from a sinusoidal excitation may be represented by a complex number, therefore possessing an actual and an imaginary component. There are two possible representations of a complex number:

The Cartesian form: a+b.i, where a is the actual part, and b is the imaginary part); and

The polar form: Aₘ^{θ} where Aₘ is the amplitude, and 0 is the phase.

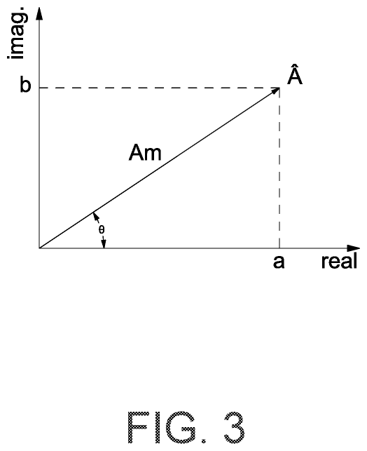

The image shown in FIG. 3 graphically shows the representation of complex number Å in Cartesian and polar forms:

The impedance formula in complex form is similar to the actual one:

$$\hat{Z} = \frac{\hat{V}}{\hat{I}}$$

Angle θ of complex number Z indicates the phase difference between tension \hat{V} and current \hat{t}. Physically, angle θ is shown as a difference between the current and tension sine waves in the measured element.

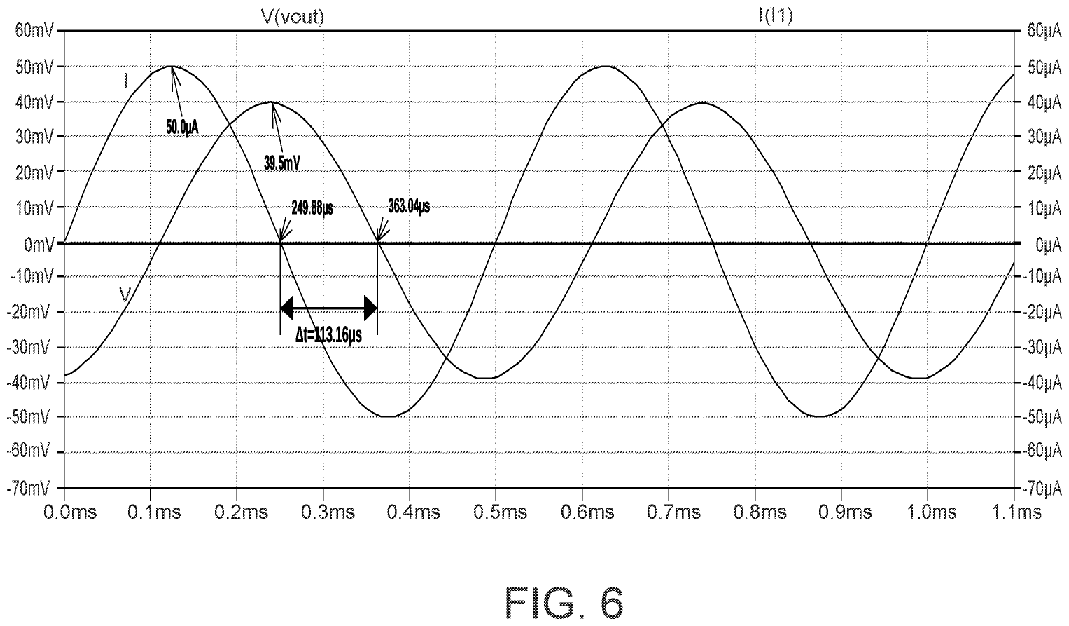

FIG. 6 shows an example of sinusoidal current excitation. Through the formulas below, the phase delay may be calculated, which in this specific case is 81.5°.

$$f = 2 kHz \\ T = \frac{1}{f} = 1 / 2000 = 500 μs \\ θ = \frac{Δ t}{T} \cdot 360 ° = \frac{113.16 μ}{500 μ} \cdot 360 ° = 81.5 °$$

With that knowledge, it may be stated that the difference between a standard Impedance audiometry equipment and the equipment proposed in this Invention patent application is the ability to measure and record difference (or phase delay) between excitation and response, in addition to amplitude.

Other Measurement Characteristics

There is extensive content in the literature indicating that fluid impedances and the human body show capacitive characteristics, in other words, the tension will be delayed regarding the current.

Tests performed by the Applicant have confirmed this expectation. Technically, when tension is delayed regarding the current, there is a negative phase impedance. When it is advanced, there is a positive phase impedance. In the human body and in liquids, tension is always delayed regarding the current, therefore, strictly speaking, a negative impedance phase would ensue. Therefore, the phase will be addressed as a “Phase delay”, and use the positive amount to facilitate the notation and the analysis.

In an objective and summarized way, the following is determined:

The human body, including mucosa, have a baseline characteristic capacitive impedance, with marked phase delay.
Liquid or bolus in the esophagus have featured a significantly different electric impedance with the phase from the characteristic organ baseline.
The electric impedance phase reading in the esophagus shall allow identification of the presence/movement of substances/bolus.
The current Impedance audiometry exam only records the impedance amplitude, and this invention patent application proposes that both the amplitude and the phase of this impedance are recorded.

Advantages of Impedance Audiometry with Phase Analysis

Impedance audiometry proposes that the impedance amplitude of the esophageal contents could be used to determine the movement of substances/bolus in this organ. The Applicant proposes that the phase (or phase delay) of impedance is used for this same purpose, but it should be noted that such parameter is shown as a more robust marker.

The Applicant has noticed that esophagus impedances, physiologically speaking, have a higher phase delay that liquids and transported substances, therefore, monitoring phase delay in different points of the esophagus, it may be implied that there is something between the electrodes. The phase has shown to be more stable than the impedance amplitude, in other words, requires less spurious variations per movement and, for this reason, some phenomena have shown with more clarity.

Analyzing both impedance magnitudes (amplitude and phase), therefore, brings the following advantages:

Facilitates analysis automation, because phenomena will become more evident and allow software to identify occurrences. Even with use of artificial intelligence software, the availability of data from different sources helps in development.
Higher accuracy. Impedance audiometry is affected by variation between examiners. With higher clarity in observation of phenomena, less subjective analyses will be generated and, therefore, diagnostic accuracy will increase. This allows determination of normality values with lower uncertainties.
Lower analysis time. Increasing phenomena clarity and analysis software accuracy, exam review by the human examiner for diagnosis elaboration will be much faster.
Lower cost. By decreasing the analysis time, the productivity of a very expensive and limited asset will be logically increased, namely the human examiner (normally a physician). In addition, accuracy will increase, and the number of inconclusive exams will decrease, thus also decreasing repeated exams and the need for complementary exams.

Considering it is a medical exam, important social benefits are also expected:

With the increased availability of the exam at a lower cost, more people will be able to carry out the procedure.
With increased accuracy, people will be treated more efficiently and faster.

### PRACTICAL EXAMPLES

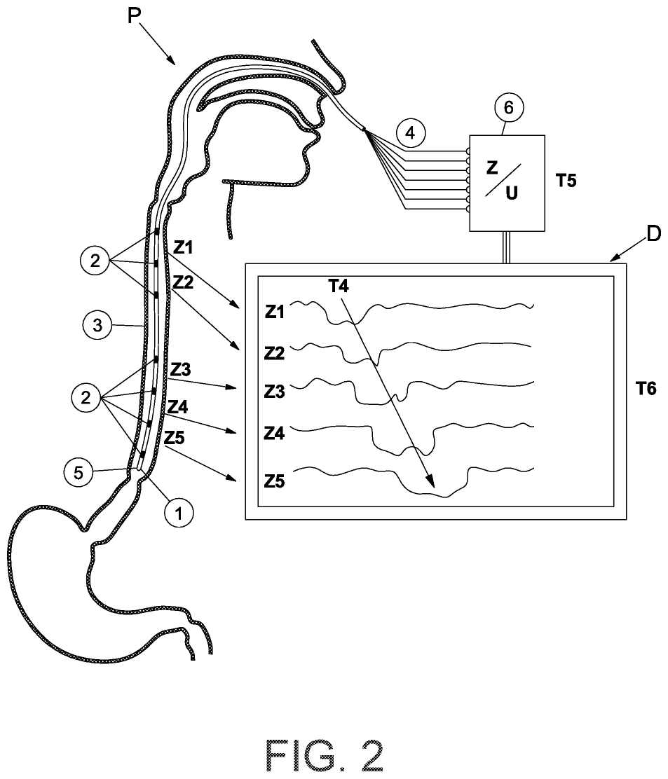

In the example shown in FIG. 2, multiple electrodes are placed along the catheter, which enables determination of the movement direction of the substance/bolus inside the esophagus of patient P.

The aforementioned FIG. 2 shows the insertion of a probe through the nostril of patient P, which is connected to an equipment that stimulates and reads the amplitude of impedances. The same FIG. 2 shows the electrodes (rings) that are connected by wires to said equipment, and the distribution of the electrodes defines mediation zones along the probe (for example, Z1 to Z5), said zones with parameters measured and shown in a display D in which the same zones are shown (Z1 to Z5), its parameter variations from which the bolus movement may be seen.

The explanation of the main diagnosis exams in the gastroenterology sub-area are featured below, where Impedance audiometry is applied.

As previously outlined, Impedance audiometry today is a widely used exam in gastroenterology diagnostics. It shall be explained below how this exam complements the other existing main exams:
pH monitoring is an exam that records pH (acidity) of the esophagus for 24 hours. Its limitation is related to the fact that this method records only acidic refluxes. When combined with Impedance audiometry (as previously mentioned, a simultaneous combined examination is performed), in which gaseous movements, non-acidic refluxes and other bolus movements that are invisible to the pH monitoring are also viewed.
Manometry is an exam in which the pressures of the esophagus and its sphincters during function. In this exam, multiple pressure sensors are placed in different parts of the esophagus in order to check the tonus of peristaltic waves, sphincter opening and closure. This exam is carried out in about 15 minutes, while the patient is awake and performing maneuvers (basically swallowing). Combined with the Impedance audiometry (a catheter/equipment capable of measuring both magnitudes simultaneously is used), it is possible to see beyond the tonus, the bolus movement and this way, better assess the functional effectiveness of the studied organs. An advantage of Impedance audiometry in this case is that saline solution or slightly saline paste may be used as markers, which are cheap substances, easily available and non-toxic.
Video Swallowing Monitoring is an exam during which a patient is offered food added of a contrast medium, and the bolus movement is continuously monitored through an x-ray equipment. It is a widely used exam, albeit with several limitations: the measurement time is short (after all, it employs X-rays), uses expensive equipment, requires use of contrast media (normally barium), which may cause allergies, among other negative aspects. The Video Swallowing Monitoring exam does not replace Impedance audiometry, or vice-versa, which are complementary exams and each with specific indications.
Endoscopy is probably the most commonly known exam in the field of Gastroenterology. Although it studies the same organs than the aforementioned exams, it has an entirely different purpose. Endoscopy enables viewing the mucosal state, structures, positioning, length, etc. In addition to examination, it is also a therapeutic technique, as procedures such as biopsy, polyp removal, cauterization and even minor surgeries may be performed; however, it is an invasive procedure and only allows analysis of structures in static condition, which prevents monitoring of organ functions.

In summary, Impedance audiometry, Manometry, pH monitoring, Video Swallowing Monitoring and Endoscopy are complementary exams regarding the study of esophageal dysfunctions. For the most part, a sub-set of these examinations will be required to carry out a correct diagnosis.

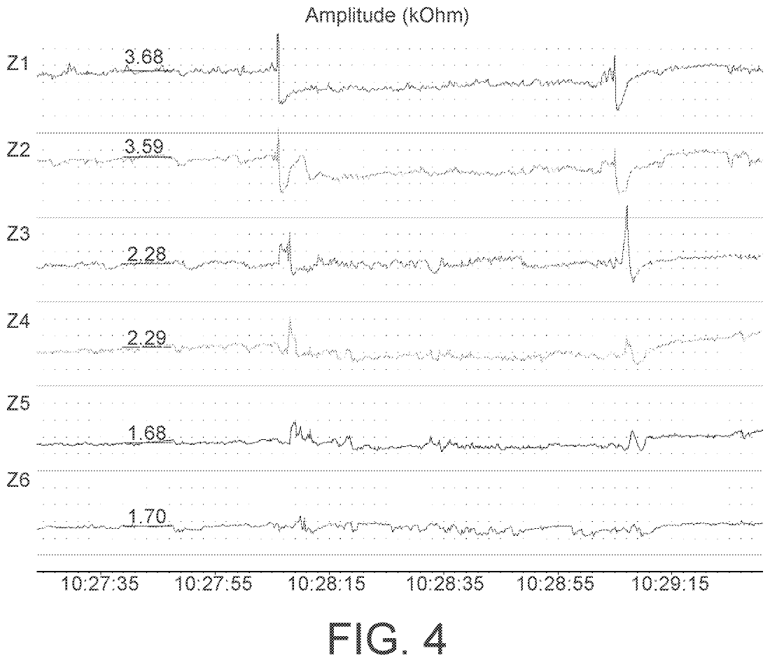

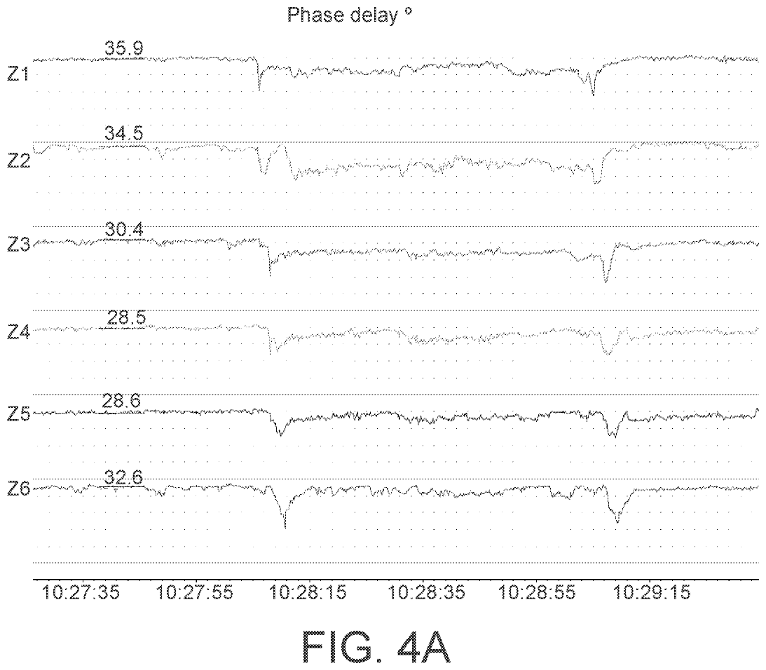

FIGS. 4 and 4A show the same phenomenon (two swallows) seen through the impedance (FIG. 4) and phase (FIG. 4A) amplitude.

The Amplitude Charts Show:

The impedance magnitude greatly varies between channels. There are 3.7 k Ohm in higher channels (near the pharynx) and 1.7 k Ohm in the lower channel (near the stomach).
Phenomena are practically imperceptible in channel Z6, confounding with natural variance.
In some channels impedance has increased, while decreasing in others.

All these factors, in other words, this magnitude variability, inconsistency (either increase or decrease), confusion with natural variations, greatly affects analysis (either automatic or human).

Now, it is possible to observe in Phase delay charts:

Magnitude has no variation, and in this example, it remains around 30° on all channels.
There was a consistent decrease in phase delay during phenomena.
Even in channel Z6, where phenomena were nearly invisible by the amplitude, it is clearly seen using the phase parameter.

Such as evidenced through joint observation and the analysis made for FIGS. 4 and 4A, uniting amplitude observations and impedance phase, certainly a diagnostic gain will be experienced.

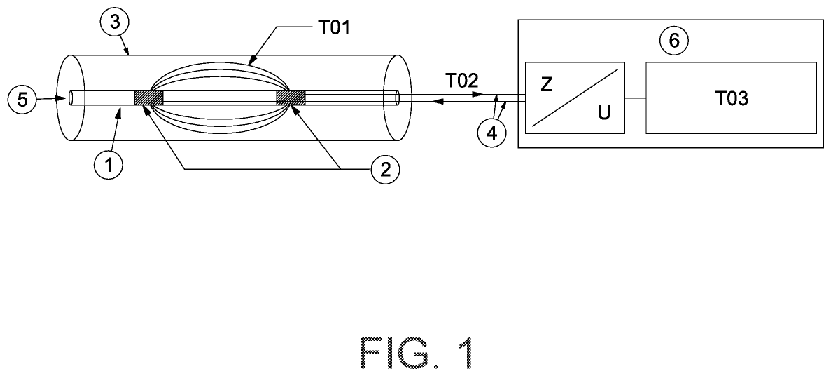

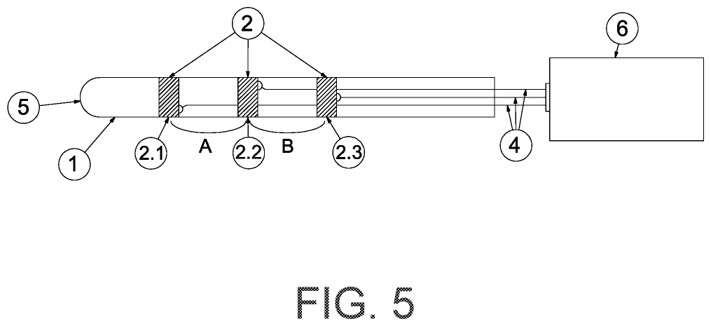

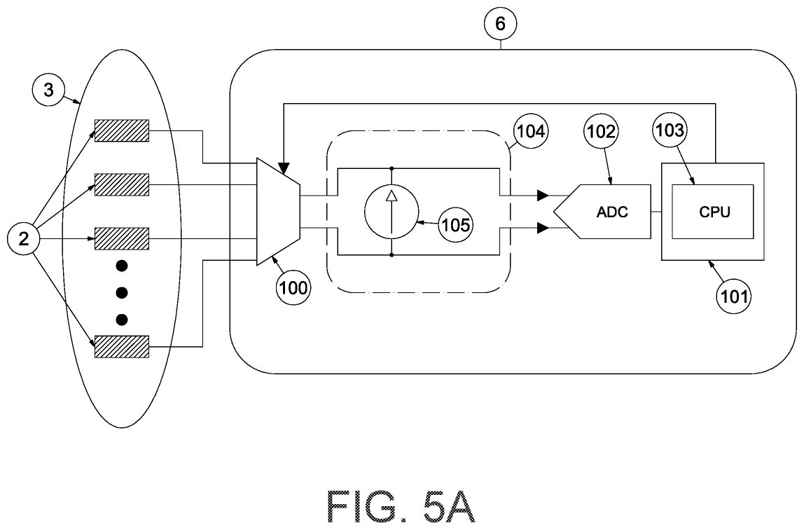

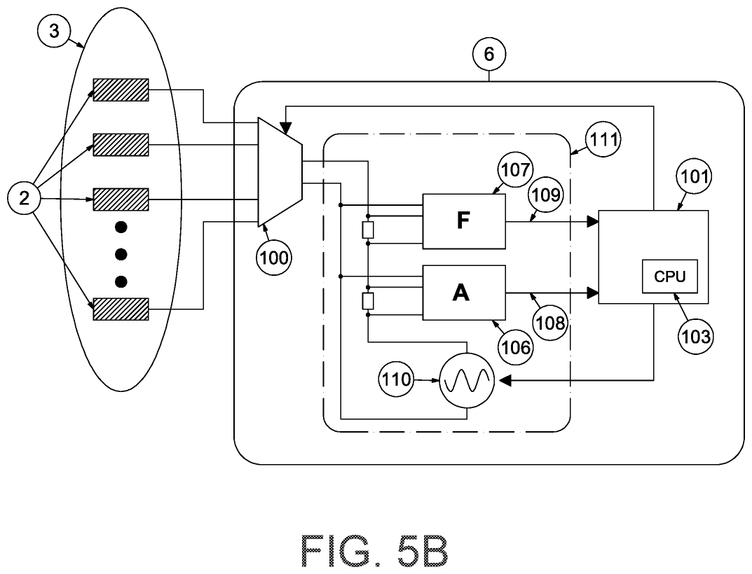

This Invention patent application also addresses the “EQUIPMENT FOR MEASURING MOVEMENT OF SUBSTANCE/BOLUS IN A TUBULAR ORGAN”, said device being part of the basic schematic shown in FIGS. 1 and 5.

FIG. 1 addresses the physical arrangement of current Impedance audiometry, in which a flexible insulating catheter (plastic) (1), including metallic rings (2), which is introduced in the organ (3) to be studied (normally the esophagus).

Said metallic rings (2) are connected by wires (4) running internally to the probe (5) defined by the flexible insulating catheter (1) and metallic rings (2) mounted therein to the equipment (6).

The equipment (6) applies electric excitation to the metallic rings (2) that act as electrodes, and this excitation shall pass through the organ (3) and its contents (after all, the catheter (1) is insulating). The equipment (6) shall measure the response to this excitation.

This is the schematic for a single channel. Normally multiple channels are used, and, in this case, the pattern is repeated, such as shown in FIG. 5. According to the proposal brought about by this Invention patent application, two adjacent channels may share one of the rings (2) in other words, it is possible that two channels are using only three rings (2): channel “A” using ring (2.1) and (2.2), channel B using rings (2.2) and (2.3).

The Applicant understands that such technical feature is an advantage, since it is possible to keep using the same catheters (1) already developed, certified and registered for use in medical Impedance audiometry examinations.

Regarding the electronic equipment system (6), the electronics for phase measurement beyond an impedance amplitude must be updated, and the application of alternate current excitation (I) is proposed; measure a response under tension (V), and calculate the impedance through the ratio between peak values of both magnitudes, such as may be understood through the chart addressed in Figure

$${Z}_{m} = \frac{{V}_{p}}{{I}_{p}} , \qquad 6$$

Vp=peak tension, Ip=peak current, Zm=impedance amplitude.

For phase calculation, normally the time between “zero crossings” of both waves is measured (the most used form, but could also be the time between peaks): The phase in degrees)(°) would be obtained through the formula: θ=Δt·f·360°, with f=excitation frequency, Δt=time between crossings.

FIG. 5A shows this equipment, in which the square wave excitation is obtained from a continuous current (CC).

The switch block (100) is used both for multiplexing between various channels and to generate the AC behavior in square wave from the CC excitation (105). The processor/microcontroller (101), connected to (or including) a Analog to Digital Converter (ADC) (102), shall digitize the tension values read and carry out all necessary calculations to extract actual and imaginary components from the impedance value. These calculations are based in the Discrete Fourier Transform (DFT).

In the same FIG. 5A, the representative block of the processor/microcontroller (101) may also be seen, the CPU block (103).

FIG. 5B shows a diagram of a more analogical equipment (6), where a sinusoidal excitation source (110) may be used, either for current or tension. This source may require some control from the processor/microcontroller (101) for synchronization.

The amplitude gauges (106) and phase gauges (107) are also featured, and require access both to current signals and tension signals. These gauges may be combined in some setups. The gauges shall convert the sinusoidal current and tension signals in impedance amplitude values (108) and phase values (109), which will then be read by the processor (101), digitalized (if still in analog format), and stored.

Normally a switch block (100) (multiplexer) is used to excite a pair of rings (2) at once, therefore an electronic block (111) is used only for multiple channels.

In some applications, the processor may have more assignments, such as controlling excitation, digitalizing analog values, measuring times or integrating mathematical calculations. In other words, the generic “phase measurement” blocks and “amplitude measurement”, the actual processor may have a higher or lower participation.

The device (6) proposed herein may be implemented within a wide range of solutions, while the model shown herein is based in a circuit capable of carrying out the measurements indicated above, but basically may use an alternate current or tension source (110), which would carry out the excitation, also featuring a phase detector, implemented analogically, digitally or in a mixed fashion.

In an alternate form based on more digital technology, excitation and response signals are digitalized, and a processor (101) is provided which would carry out all calculations using the obtained samples.

There are countless forms of measuring phases, and there are also integrated circuits dedicated to such objective.

Necessary modifications in an Impedance audiometry equipment (6) for impedance phase reading may vary greatly according to implementation.

An equipment (6) based on a more analogical approach would probably require a change (redesign) to include circuits capable of determining the phase. A more digital equipment could accomplish such condition through a firmware update.

Methodology Used in the Equipment Proposed Herein

In the equipment proposed herein, a digital approach for impedance reading is used.

Simpler circuits (104) are used to carry out excitation with a higher workload assigned to a processor (101) that digitalizes and processes the samples in order to extract its features.

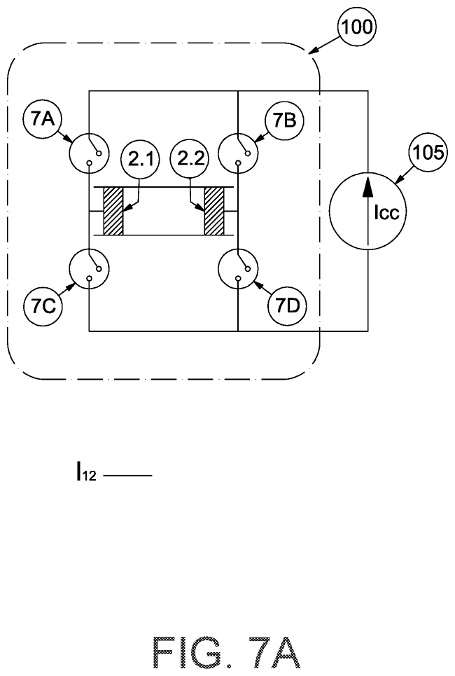

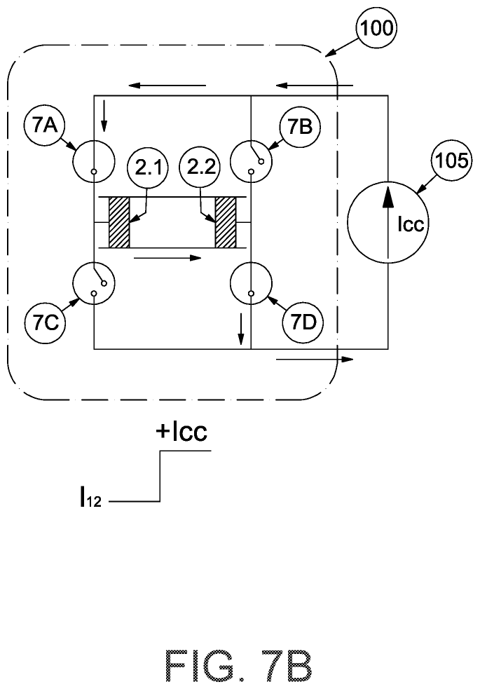

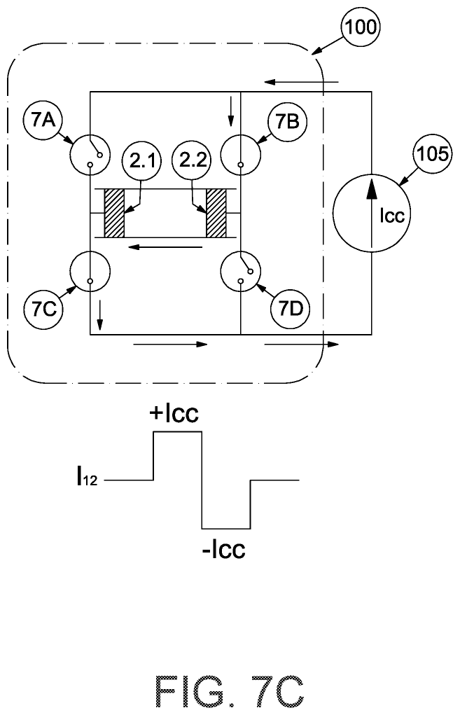

Alternate square current excitation is used, said current ranging between 1 Khz to 10 KHz. This excitation is generated by a continuous Icc current source (105) and applied to the rings 92) through a set of analog switches (7) ((7A), (7B), (7C) and (7D)), which are part of the switch block (100), in order to make it flow either on one direction or the other, thus creating the alternate behavior, as shown in FIGS. 7A, 7B and 7C.

FIGS. 7A, 7B and 7C, when all switches (7) are open, no current passes between the rings (2) (zero current), such as shown in FIG. 7A; when switches (7A) and (7D) are closed, the Icc current generated by the source (105) passes from ring (2.1) to ring (2.2); and when switches (7B) and (7C) are closed, the Icc current passes from ring (2.2) to ring (2.1).

As a result, current I₁₂ varies between +Icc and—Icc. These switches (7) shall be under control of the microprocessor (101) of the equipment (6). This is a very convenient way of generating a current excitation in an alternate square wave form.

The schematic described above that is shown in FIGS. 7A, 7B and 7C may be replicated to multiple channels, therefore with two switches being required for each channel.

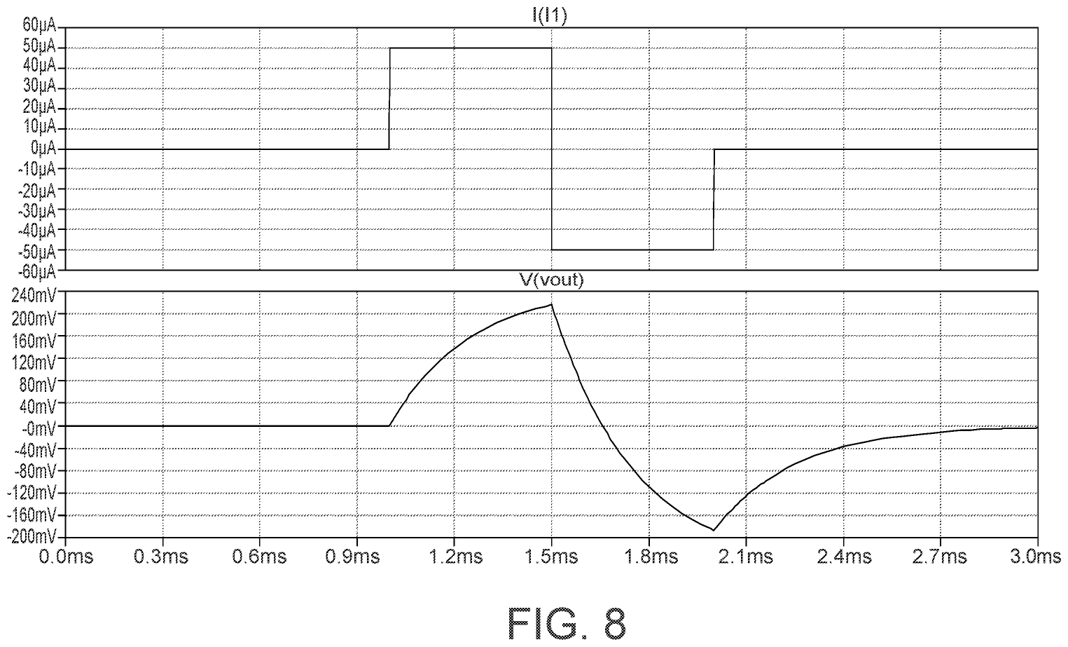

When a load with capacitive characteristics is excited using a current as described, a wave form is obtained in the shape shown in FIG. 8.

In order to obtain the sinusoidal amplitude from this waveform, the Discrete Fourier Transform (DFT) is applied. Current microprocessors have sufficient calculation power for this task.

Thus, the tension signal obtained from the excitation is digitalized, and the DFT fundamental coefficient is calculated. An interesting feature is that DFT naturally provides a complex number in rectangular form.

Therefore, conveniently, due to the method used in the equipment (6) that is object of this Invention patent application for calculation of impedance, the complex value is already known.

Previously, regarding the state of the art, only the amplitude was calculated and stored, while the phase was disregarded.

An update in the firmware (processor (101) program) was required for equipment (6) in order to store the full complex value.

It should be noted that, in practical terms, in order to stabilize readings, three consecutive excitations are carried out: 500 us at OV (first under tension) to outflow electric charges that may have formed in the electrodes (rings (2)), followed by a current excitation for polarization and, lastly, an excitation for measurement.

In summary, as the method proposed in this patient application for impedance calculation is strongly digitalized, no physical changes in the equipment (6) were required, but a change in programming.

Applicant has no knowledge of any other equipment that uses this technique for impedance measurement.

What is claimed is:

1. A method for measuring movement of substance/bolus in a tubular organ, wherein said method is based on the combined measurement of amplitude and impedance phase, said tubular organ being mainly the esophagus; the proposed method takes the difference between tension and current (impedance phase) as a parameter in the Impedance audiometry exam, from an excited element with alternate current; the proposed method uses a probe (5) defined by a flexible insulating catheter (1), including metallic rings (2) that is introduced in the organ 93) to be examined; the metallic rings (2) are connected by wires (4) that run internally through the probe (5) defined by the flexible insulating catheter (1); the probe (5) is connected to an equipment (6) that applies an electric excitation to the metallic rings (2) which act as electrodes, this excitation passes through the organ (3) and its contents; the equipment (6) measures the response to this excitation, showing the results in a display (D); the equipment (6) proceeds to the reading/storage/display of impedance amplitude values and also impedance phase.

2. A equipment for measuring movement of substance/bolus in a tubular organ, to be used in the method defined in claim 1, wherein it is connected to a probe (5) defined by a flexible insulating catheter (1), comprising metallic rings (2); said metallic rings 92) are connected by wires (4) that run internally through the probe (5) defined by the flexible insulating catheter (1); the probe (5) is connected to the equipment (6); the metallic rings (2) may be positioned so that two adjacent channels may share a ring (2); the equipment (6) shows the data on a display (D).

3. The equipment for measuring movement of substance/bolus in a tubular organ, according to claim 2, wherein it includes a switch block (100) to carry out multiplexing between various channels and also generating an AC behavior in square wave from the CC excitation (105) associated to a circuit (104); the equipment (6) includes a processor/microcontroller (101) and an analog to Digital Converter (ADC) (102), which digitalizes tension values read and carries out all necessary calculations to extract actual and imaginary components of the impedance value; the equipment (6) includes a CPU (103).

4. The equipment for measuring movement of substance/bolus in a tubular organ, according to claim 3, wherein the calculations are based in the Discrete Fourier Transform (DFT).

5. The equipment for measuring movement of substance/bolus in a tubular organ, according to claim 2, wherein the equipment (6) includes amplitude gauges (106) and phase gauges (107), which access both current and tension signals; the gauges convert sinusoidal current and tension signals in impedance amplitude values (108) and phase values (109), which will then be read by the processor 9101), digitalized and stored; the switch block (multiplexer) 9100) is used to excite a pair of rings (2) at one time, whereas an electronic block (111) is used for multiple channels; the equipment (6) includes a sinusoidal excitation (110), either for current or tension, which may be controlled by the processor/microcontroller (101) for synchronization.

6. The equipment for measuring movement of substance/bolus in a tubular organ, according to claim 2, wherein the square alternated current excitation is generated with a 1 Khz to 10 Khz frequency, by a continuous current source Icc (105), and applied to the rings (2) through a set of analog switches (7)((7A), (7B), (7C) and (7D)), which are part of the switch block (100), inducing flow in alternate directions, thus creating the alternated behavior.

7. The equipment for measuring movement of substance/bolus in a tubular organ, according to claim 6, wherein the continuous current excitation is replaced by a constant tension source.
